(19) United States
(12) Patent Application Publication
(10) Pub. No.: US 2023/0041745 A1
(43) Pub. Date: Feb. 9, 2023

(54) Telehealth Assistance System and Method

(71) Applicant: Nuance Communications, Inc., Burlington, MA (US)

(72) Inventor: Eduardo Olvera, Phoenix, AZ (US)

(21) Appl. No.: 17/554,817

(22) Filed: Dec. 17, 2021

Related U.S. Application Data
(60) Provisional application No. 63/230,492, filed on Aug. 6, 2021.

(51) Int. Cl. G16H 40/67; G16H 10/20; G16H 10/60; G16H 80/00

(52) U.S. Cl. CPC G16H 40/67; G16H 10/20; G16H 10/60; G16H 80/00

(57) ABSTRACT

A method, computer program product, and computing system for receiving a notification that a patient has arrived to a telehealth session before the telehealth session begins. The notification is received via a computing device. In response to receiving the notification that the patient has arrived to the telehealth session before the telehealth session begins, information associated with the patient is automatically pulled by a virtual assistant. The patient is prompted by the virtual assistant to complete a task before the telehealth session begins. A question is received from the patient before the telehealth session begins. Patient data may be obtained from one or more sources. The obtained patient data is processed to determine if the patient data is indicative of a possible medical condition and the medical condition is provided to a medical professional. An answer to the question is provided. The answer is personalized to the patient.

### RELATED APPLICATION(S)

This application claims the benefit of U.S. Provisional Application No. 63/230,492, filed on 6 Aug. 2021, the entire contents of which are herein incorporated by reference.

### BACKGROUND

When it comes to an appointment with a medical professional, the appointment may take several forms, including in a telehealth appointment. However, in a telehealth appointment, a healthcare professional may be jeopardized by discoordination, technical difficulties, and/or challenges in communication and appointment administration before and/or during a telehealth appointment.

### SUMMARY OF DISCLOSURE

In one implementation, a computer-implemented method, performed by one or more computing devices, may include but is not limited to receiving a notification that a patient has arrived to a telehealth session on a virtualized platform before the telehealth session begins. The notification may be received via a computing device. In response to receiving the notification that the patient has arrived to the telehealth session before the telehealth session begins, information associated with the patient may be automatically pulled by a virtual assistant. The patient may be prompted by the virtual assistant to complete a task before the telehealth session begins. A question may be received from the patient before the telehealth session begins. Patient data may be obtained from one or more sources. The one or more sources may include at least one of a patient database, a machine vision system that is configured to provide an analysis of a physical attribute of the patient, and an audio recording system that is configured to provide an analysis of an auditory attribute of the patient. The obtained patient data may be processed to determine if the patient data is indicative of a possible medical condition. If a medical condition is determined to be present, the medical condition may be provided to a medical professional. An answer to the question received from the patient may be provided. The answer may be personalized to the patient.

One or more of the following features may be included. The task to be completed by the patient before the telehealth session begins may include one or more of a fillable form and an interactive symptom checker. The interactive symptom checker may include an interactive scale where the patient may indicate a current physical pain level. The question the patient asks the virtual assistant may include one or more of a question regarding which medical provider the telehealth appointment will be with, a question regarding the patient's insurance, and a question regarding past patient data recorded during a previous telehealth visit. The obtained patient data may be compared to the past patient data recorded in the previous telehealth session. It may be determined if one or more of a medical condition is present and if a previously identified medical condition is present based at least in part on the obtained patient data and the past patient data. The auditory attribute of the patient may include a cough recorded by the audio recording system after the patient has arrived to the telehealth session and before the telehealth session begins. It may be determined that the virtual assistant cannot answer the question from the patient. In response to determining that the virtual assistant cannot answer the question from the patient, the question may be provided to the medical professional when the telehealth session begins.

In another implementation, a computer program product may reside on a computer readable storage medium having a plurality of instructions stored thereon which, when executed across one or more processors, may cause at least a portion of the one or more processors to perform operations that may include but are not limited to receiving a notification that a patient has arrived to a telehealth session on a virtualized platform before the telehealth session begins. The notification may be received via a computing device. In response to receiving the notification that the patient has arrived to the telehealth session before the telehealth session begins, information associated with the patient may be automatically pulled by a virtual assistant. The patient may be prompted by the virtual assistant to complete a task before the telehealth session begins. A question may be received from the patient before the telehealth session begins. Patient data may be obtained from one or more sources. The one or more sources may include at least one of a patient database, a machine vision system that is configured to provide an analysis of a physical attribute of the patient, and an audio recording system that is configured to provide an analysis of an auditory attribute of the patient. The obtained patient data may be processed to determine if the patient data is indicative of a possible medical condition. If a medical condition is determined to be present, the medical condition may be provided to a medical professional. An answer to the question received from the patient may be provided. The answer may be personalized to the patient.

One or more of the following features may be included. The task to be completed by the patient before the telehealth session begins may include one or more of a fillable form and an interactive symptom checker. The interactive symptom checker may include an interactive scale where the patient may indicate a current physical pain level. The question the patient asks the virtual assistant may include one or more of a question regarding which medical provider the telehealth appointment will be with, a question regarding the patient's insurance, and a question regarding past patient data recorded during a previous telehealth visit. The obtained patient data may be compared to the past patient data recorded in the previous telehealth session. It may be determined if one or more of a medical condition is present and if a previously identified medical condition is present based at least in part on the obtained patient data and the past patient data. The auditory attribute of the patient may include a cough recorded by the audio recording system after the patient has arrived to the telehealth session and before the telehealth session begins. It may be determined that the virtual assistant cannot answer the question from the patient. In response to determining that the virtual assistant cannot answer the question from the patient, the question may be provided to the medical professional when the telehealth session begins.

In another implementation, a computing system may include one or more processors and one or more memories configured to perform operations that may include but are not limited to receiving a notification that a patient has arrived to a telehealth session on a virtualized platform before the telehealth session begins. The notification may be received via a computing device. In response to receiving the notification that the patient has arrived to the telehealth session before the telehealth session begins, information associated with the patient may be automatically pulled by a virtual assistant. The patient may be prompted by the virtual assistant to complete a task before the telehealth session begins. A question may be received from the patient before the telehealth session begins. Patient data may be obtained from one or more sources. The one or more sources may include at least one of a patient database, a machine vision system that is configured to provide an analysis of a physical attribute of the patient, and an audio recording system that is configured to provide an analysis of an auditory attribute of the patient. The obtained patient data may be processed to determine if the patient data is indicative of a possible medical condition. If a medical condition is determined to be present, the medical condition may be provided to a medical professional. An answer to the question received from the patient may be provided. The answer may be personalized to the patient.

One or more of the following features may be included. The task to be completed by the patient before the telehealth session begins may include one or more of a fillable form and an interactive symptom checker. The interactive symptom checker may include an interactive scale where the patient may indicate a current physical pain level. The question the patient asks the virtual assistant may include one or more of a question regarding which medical provider the telehealth appointment will be with, a question regarding the patient's insurance, and a question regarding past patient data recorded during a previous telehealth visit. The obtained patient data may be compared to the past patient data recorded in the previous telehealth session. It may be determined if one or more of a medical condition is present and if a previously identified medical condition is present based at least in part on the obtained patient data and the past patient data. The auditory attribute of the patient may include a cough recorded by the audio recording system after the patient has arrived to the telehealth session and before the telehealth session begins. It may be determined that the virtual assistant cannot answer the question from the patient. In response to determining that the virtual assistant cannot answer the question from the patient, the question may be provided to the medical professional when the telehealth session begins.

The details of one or more implementations are set forth in the accompanying drawings and the description below. Other possible example features and/or possible example advantages will become apparent from the description, the drawings, and the claims. Some implementations may not have those possible example features and/or possible example advantages, and such possible example features and/or possible example advantages may not necessarily be required of some implementations.

### BRIEF DESCRIPTION OF THE DRAWINGS

FIG. 1 is an example diagrammatic view of a telehealth assistance process coupled to a distributed computing network according to one or more example implementations of the disclosure;

FIG. 2 is an example diagrammatic view of a modular ACD system incorporating the telehealth assistance process of FIG. 1 according to one or more example implementations of the disclosure;

FIG. 3 is an example diagrammatic view of a mixed-media ACD device included within the modular ACD system of FIG. 2 according to one or more example implementations of the disclosure;

FIG. 4 is an example flow chart of one implementation of the telehealth assistance process of FIG. 1 according to one or more example implementations of the disclosure; and

FIGS. 5a-5c are example diagrammatic views of a telehealth assistance process according to one or more example implementations of the disclosure.

Like reference symbols in the various drawings indicate like elements.

### DETAILED DESCRIPTION OF THE PREFERRED EMBODIMENTS

### System Overview:

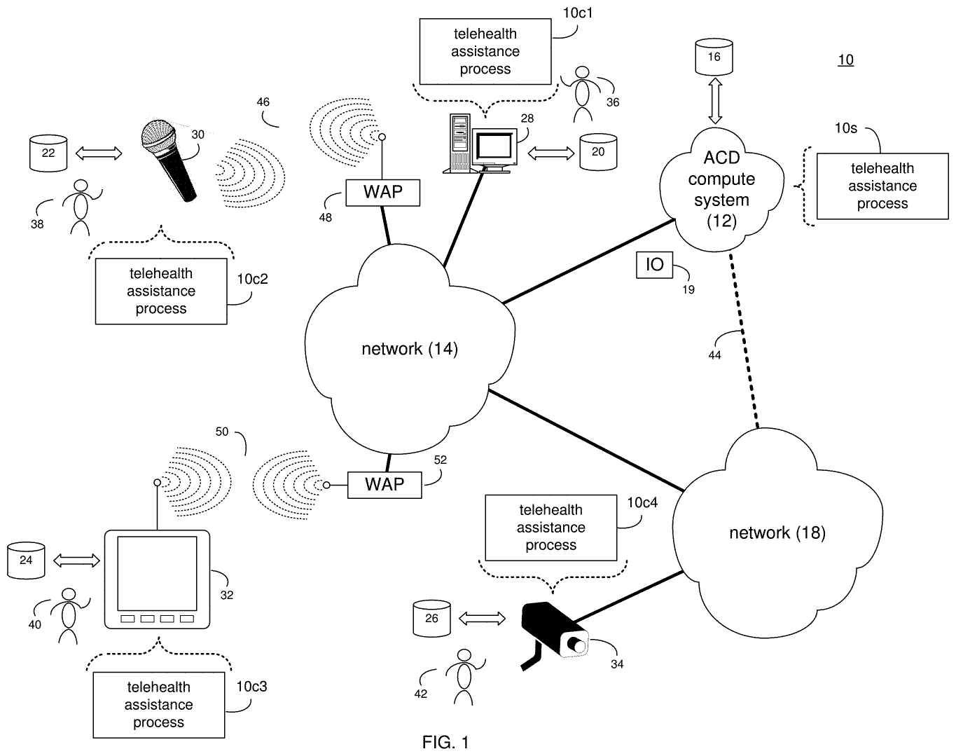

Referring to FIG. 1, there is shown telehealth assistance process 10. Telehealth assistance process 10 may be implemented as a server-side process, a client-side process, or a hybrid server-side/client-side process. For example, telehealth assistance process 10 may be implemented as a purely server-side process via telehealth assistance process 10s. Alternatively, telehealth assistance process 10 may be implemented as a purely client-side process via one or more of telehealth assistance process 10c1, telehealth assistance process 10c2, telehealth assistance process 10c3, and telehealth assistance process 10c4. Alternatively still, telehealth assistance process 10 may be implemented as a hybrid server-side/client-side process via telehealth assistance process 10s in combination with one or more of telehealth assistance process 10c1, telehealth assistance process 10c2, telehealth assistance process 10c3, and telehealth assistance process 10c4.

Accordingly, telehealth assistance process 10 as used in this disclosure may include any combination of telehealth assistance process 10s, telehealth assistance process 10c1, telehealth assistance process 10c2, telehealth assistance process 10c3, and telehealth assistance process 10c4.

Telehealth assistance process 10s may be a server application and may reside on and may be executed by automated clinical documentation (ACD) compute system 12, which may be connected to network 14 (e.g., the Internet or a local area network). ACD compute system 12 may include various components, examples of which may include but are not limited to: a personal computer, a server computer, a series of server computers, a mini computer, a mainframe computer, one or more Network Attached Storage (NAS) systems, one or more Storage Area Network (SAN) systems, one or more Platform as a Service (PaaS) systems, one or more Infrastructure as a Service (IaaS) systems, one or more Software as a Service (SaaS) systems, edge computing systems (e.g., where some of the processing happens in end nodes, like audio capturing devices themselves, before passing the post-processed information to another system for processing), a cloud-based computational system, and a cloud-based storage platform.

As is known in the art, a SAN may include one or more of a personal computer, a server computer, a series of server computers, a mini computer, a mainframe computer, a RAID device and a NAS system. The various components of ACD compute system 12 may execute one or more operating systems, examples of which may include but are not limited to: Microsoft Windows Server™; Redhat Linux™, Unix, or a custom operating system, for example.

The instruction sets and subroutines of telehealth assistance process 10s, which may be stored on storage device 16 coupled to ACD compute system 12, may be executed by one or more processors (not shown) and one or more memory architectures (not shown) included within ACD compute system 12. Examples of storage device 16 may include but are not limited to: a hard disk drive; a RAID device; a random access memory (RAM); a read-only memory (ROM); and all forms of flash memory storage devices.

Network 14 may be connected to one or more secondary networks (e.g., network 18), examples of which may include but are not limited to: an edge-based network, a local area network; a wide area network; or an intranet, for example.

Various IO requests (e.g. IO request 20) may be sent from telehealth assistance process 10s, telehealth assistance process 10c1, telehealth assistance process 10c2, telehealth assistance process 10c3 and/or telehealth assistance process 10c4 to ACD compute system 12. Examples of IO request 20 may include but are not limited to data write requests (i.e. a request that content be written to ACD compute system 12) and data read requests (i.e. a request that content be read from ACD compute system 12).

The instruction sets and subroutines of telehealth assistance process 10c1, telehealth assistance process 10c2, telehealth assistance process 10c3 and/or telehealth assistance process 10c4, which may be stored on storage devices 20, 22, 24, 26 (respectively) coupled to ACD client electronic devices 28, 30, 32, 34 (respectively), may be executed by one or more processors (not shown) and one or more memory architectures (not shown) incorporated into ACD client electronic devices 28, 30, 32, 34 (respectively). Storage devices 20, 22, 24, 26 may include but are not limited to: hard disk drives; optical drives; RAID devices; random access memories (RAM); read-only memories (ROM), and all forms of flash memory storage devices. Examples of ACD client electronic devices 28, 30, 32, 34 may include, but are not limited to, personal computing device 28 (e.g., a smart phone, a personal digital assistant, a laptop computer, a notebook computer, and a desktop computer), audio input device 30 (e.g., a handheld microphone, a lapel microphone, an embedded microphone (such as those embedded within IoT devices, eyeglasses, smart phones, tablet computers and/or watches) and an audio recording device), display device 32 (e.g., a tablet computer, a computer monitor, and a smart television), machine vision input device 34 (e.g., an RGB imaging system, an infrared imaging system, an ultraviolet imaging system, a laser imaging system, a SONAR imaging system, a RADAR imaging system, and a thermal imaging system), a hybrid device (e.g., a single device that includes the functionality of one or more of the above-references devices; not shown), an audio rendering device (e.g., a speaker system, a headphone system, or an earbud system; not shown), various medical devices (e.g., medical imaging equipment, heart monitoring machines, body weight scales, body temperature thermometers, and blood pressure machines; not shown), and a dedicated network device (not shown).

Users 36, 38, 40, 42 may access ACD compute system 12 directly through network 14 or through secondary network 18. Further, ACD compute system 12 may be connected to network 14 through secondary network 18, as illustrated with link line 44.

The various ACD client electronic devices (e.g., ACD client electronic devices 28, 30, 32, 34) may be directly or indirectly coupled to network 14 (or network 18). For example, personal computing device 28 is shown directly coupled to network 14 via a hardwired network connection. Further, machine vision input device 34 is shown directly coupled to network 18 via a hardwired network connection. Audio input device 30 is shown wirelessly coupled to network 14 via wireless communication channel 46 established between audio input device 30 and wireless access point (i.e., WAP) 48, which is shown directly coupled to network 14. WAP 48 may be, for example, an IEEE 802.11a, 802.11b, 802.11g, 802.11n, Wi-Fi, and/or Bluetooth device that is capable of establishing wireless communication channel 46 between audio input device 30 and WAP 48. Display device 32 is shown wirelessly coupled to network 14 via wireless communication channel 50 established between display device 32 and WAP 52, which is shown directly coupled to network 14.

The various ACD client electronic devices (e.g., ACD client electronic devices 28, 30, 32, 34) may each execute an operating system, examples of which may include but are not limited to Microsoft Windows™, Apple Macintosh™, Redhat Linux™, or a custom operating system, wherein the combination of the various ACD client electronic devices (e.g., ACD client electronic devices 28, 30, 32, 34) and ACD compute system 12 may form modular ACD system 54.

### The Automated Clinical Documentation System:

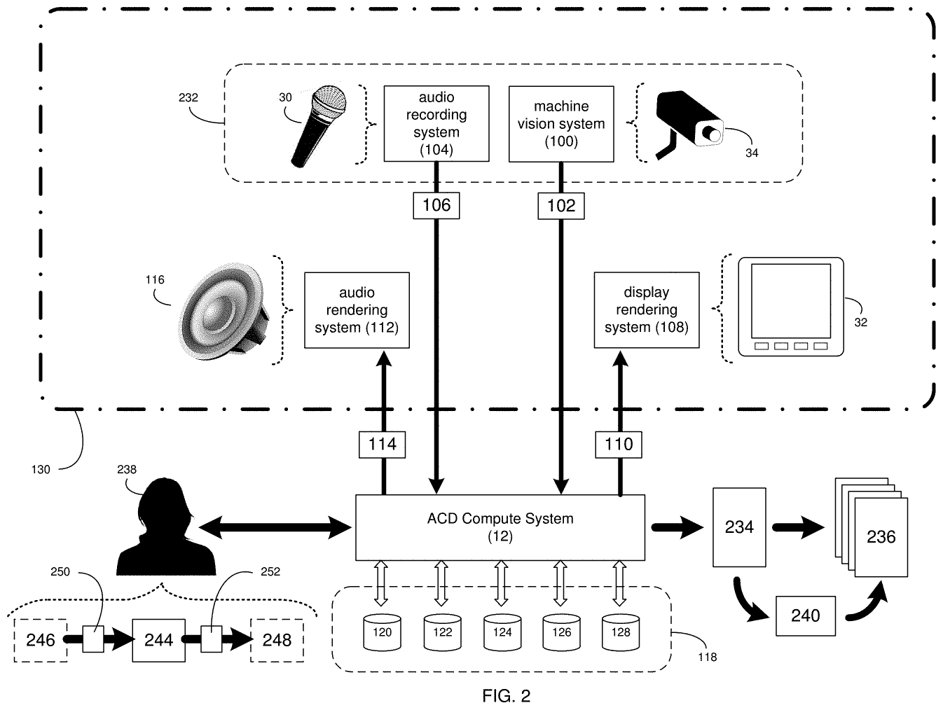

Referring also to FIG. 2, there is shown a simplified exemplary embodiment of modular ACD system 54 that is configured to automate clinical documentation. Modular ACD system 54 may include: machine vision system 100 configured to obtain machine vision encounter information 102 concerning a patient encounter; audio recording system 104 configured to obtain audio encounter information 106 concerning the patient encounter; and a compute system (e.g., ACD compute system 12) configured to receive machine vision encounter information 102 and audio encounter information 106 from machine vision system 100 and audio recording system 104 (respectively). Modular ACD system 54 may also include: display rendering system 108 configured to render visual information 110; and audio rendering system 112 configured to render audio information 114, wherein ACD compute system 12 may be configured to provide visual information 110 and audio information 114 to display rendering system 108 and audio rendering system 112 (respectively).

Example of machine vision system 100 may include but are not limited to: one or more ACD client electronic devices (e.g., ACD client electronic device 34, examples of which may include but are not limited to an RGB imaging system, an infrared imaging system, a ultraviolet imaging system, a laser imaging system, a SONAR imaging system, a RADAR imaging system, and a thermal imaging system). Examples of audio recording system 104 may include but are not limited to: one or more ACD client electronic devices (e.g., ACD client electronic device 30, examples of which may include but are not limited to a handheld microphone, a lapel microphone, an embedded microphone (such as those embedded within eyeglasses, smart phones, tablet computers and/or watches) and an audio recording device). For clarification, in some implementations, it will be appreciated that the audio recording system/device may also be considered merely as an audio capturing system/device, such as a device capable of capturing audio for a live audio streaming system/device. As such, the use of the term “recording” should not be interpreted as (necessarily) excluding a live audio stream or interpreted as (necessarily) being audio from a past event. Examples of display rendering system 108 may include but are not limited to: one or more ACD client electronic devices (e.g., ACD client electronic device 32, examples of which may include but are not limited to a tablet computer, a computer monitor, and a smart television). Examples of audio rendering system 112 may include but are not limited to: one or more ACD client electronic devices (e.g., audio rendering device 116, examples of which may include but are not limited to a speaker system, a headphone system, and an earbud system).

As will be discussed below in greater detail, ACD compute system 12 may be configured to access one or more datasources 118 (e.g., plurality of individual datasources 120, 122, 124, 126, 128), examples of which may include but are not limited to one or more of a user profile datasource, a voice print datasource, a voice characteristics datasource (e.g., for adapting the automated speech recognition models), a face print datasource, a humanoid shape datasource, an utterance identifier datasource, a wearable token identifier datasource, an interaction identifier datasource, a medical conditions symptoms datasource, a prescriptions compatibility datasource, a medical insurance coverage datasource, and a home healthcare datasource. While in this particular example, five different examples of datasources 118, are shown, this is for illustrative purposes only and is not intended to be a limitation of this disclosure, as other configurations are possible and are considered to be within the scope of this disclosure.

As will be discussed below in greater detail, modular ACD system 54 may be configured to monitor a monitored space (e.g., monitored space 130) on a telehealth platform (e.g., telehealth platform 500), where a telehealth platform (e.g., telehealth platform 500) is a digital platform that allows a patient to remotely access via a client electronic device (e.g., client electronic devices 28, 30, 32, 34) to manage the patient's healthcare. On a telehealth platform (e.g., telehealth platform 500), a patient may be connected with a medical professional for example, but not limited to, a scheduled session between the patient and the medical professional.

Machine vision system 100 may include a plurality of discrete machine vision systems when the above-described clinical environment is larger or a higher level of resolution is desired. As discussed above, examples of machine vision system 100 may include but are not limited to: one or more ACD client electronic devices (e.g., ACD client electronic device 34, examples of which may include but are not limited to an RGB imaging system, an infrared imaging system, an ultraviolet imaging system, a laser imaging system, a SONAR imaging system, a RADAR imaging system, and a thermal imaging system). Accordingly, machine vision system 100 may include one or more of each of an RGB imaging system, an infrared imaging systems, an ultraviolet imaging systems, a laser imaging system, a SONAR imaging system, a RADAR imaging system, and a thermal imaging system.

Audio recording system 104 may include a plurality of discrete audio recording systems when the above-described clinical environment is larger or a higher level of resolution is desired. As discussed above, examples of audio recording system 104 may include but are not limited to: one or more ACD client electronic devices (e.g., ACD client electronic device 30, examples of which may include but are not limited to a handheld microphone, a lapel microphone, an embedded microphone (such as those embedded within eyeglasses, smart phones, tablet computers and/or watches) and an audio recording device). Accordingly, audio recording system 104 may include one or more of each of a handheld microphone, a lapel microphone, an embedded microphone (such as those embedded within eyeglasses, smart phones, tablet computers and/or watches) and an audio recording device.

Display rendering system 108 may include a plurality of discrete display rendering systems when the above-described clinical environment is larger or a higher level of resolution is desired. As discussed above, examples of display rendering system 108 may include but are not limited to: one or more ACD client electronic devices (e.g., ACD client electronic device 32, examples of which may include but are not limited to a tablet computer, a computer monitor, and a smart television). Accordingly, display rendering system 108 may include one or more of each of a tablet computer, a computer monitor, and a smart television.

Audio rendering system 112 may include a plurality of discrete audio rendering systems when the above-described clinical environment is larger or a higher level of resolution is desired. As discussed above, examples of audio rendering system 112 may include but are not limited to: one or more ACD client electronic devices (e.g., audio rendering device 116, examples of which may include but are not limited to a speaker system, a headphone system, or an earbud system). Accordingly, audio rendering system 112 may include one or more of each of a speaker system, a headphone system, or an earbud system.

ACD compute system 12 may include a plurality of discrete compute systems. As discussed above, ACD compute system 12 may include various components, examples of which may include but are not limited to: a personal computer, a server computer, a series of server computers, a mini computer, a mainframe computer, one or more Network Attached Storage (NAS) systems, one or more Storage Area Network (SAN) systems, one or more Platform as a Service (PaaS) systems, one or more Infrastructure as a Service (IaaS) systems, one or more Software as a Service (SaaS) systems, a cloud-based computational system, and a cloud-based storage platform. Accordingly, ACD compute system 12 may include one or more of each of a personal computer, a server computer, a series of server computers, a mini computer, a mainframe computer, one or more Network Attached Storage (NAS) systems, one or more Storage Area Network (SAN) systems, one or more Platform as a Service (PaaS) systems, one or more Infrastructure as a Service (IaaS) systems, one or more Software as a Service (SaaS) systems, a cloud-based computational system, and a cloud-based storage platform.

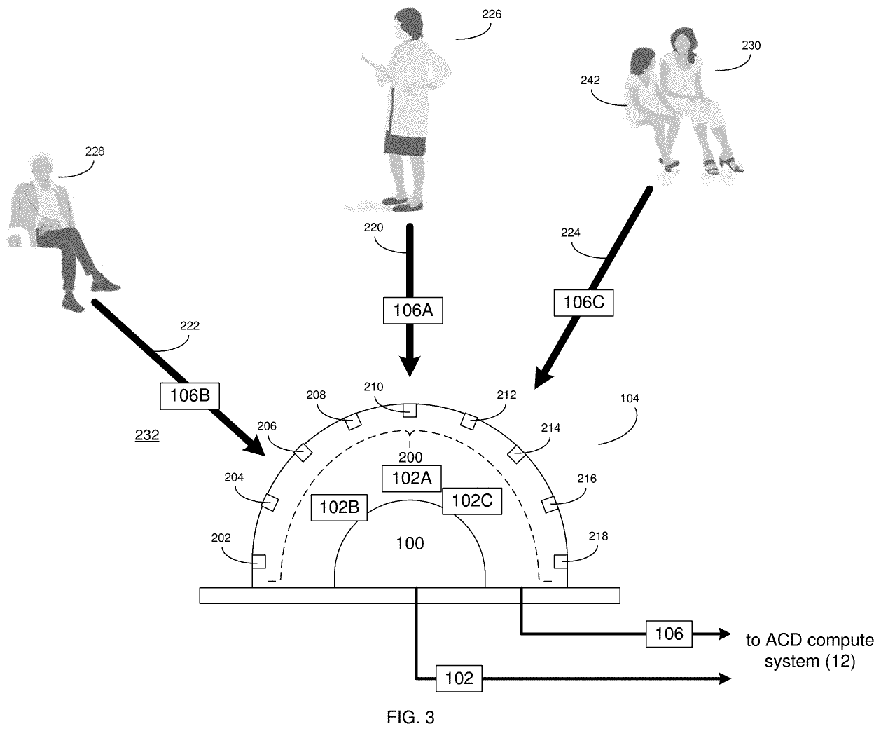

Referring also to FIG. 3, audio recording system 104 may include directional microphone array 200 having a plurality of discrete microphone assemblies. For example, audio recording system 104 may include a plurality of discrete audio acquisition devices (e.g., audio acquisition devices 202, 204, 206, 208, 210, 212, 214, 216, 218) that may form microphone array 200. As will be discussed below in greater detail, modular ACD system 54 may be configured to form one or more audio recording beams (e.g., audio recording beams 220, 222, 224) via the discrete audio acquisition devices (e.g., audio acquisition devices 202, 204, 206, 208, 210, 212, 214, 216, 218) included within audio recording system 104.

For example, modular ACD system 54 may be further configured to steer the one or more audio recording beams (e.g., audio recording beams 220, 222, 224) toward one or more encounter participants (e.g., encounter participants 226, 228, 230) of the above-described patient encounter. Examples of the encounter participants (e.g., encounter participants 226, 228, 230) may include but are not limited to: medical professionals such as doctors, nurses, physician's assistants, lab technicians, physical therapists, scribes (e.g., a transcriptionist) and/or staff members involved in the patient encounter), patients (e.g., people that are visiting the above-described clinical environments for the patient encounter), and third parties (e.g., friends of the patient, relatives of the patient and/or acquaintances of the patient that are involved in the patient encounter).

Accordingly, modular ACD system 54 and/or audio recording system 104 may be configured to utilize one or more of the discrete audio acquisition devices (e.g., audio acquisition devices 202, 204, 206, 208, 210, 212, 214, 216, 218) to form an audio recording beam. For example, modular ACD system 54 and/or audio recording system 104 may be configured to utilize audio acquisition device 210 to form audio recording beam 220, thus enabling the capturing of audio (e.g., speech) produced by encounter participant 226 (as audio acquisition device 210 is pointed to (i.e., directed toward) encounter participant 226). Additionally, modular ACD system 54 and/or audio recording system 104 may be configured to utilize audio acquisition devices 204, 206 to form audio recording beam 222, thus enabling the capturing of audio (e.g., speech) produced by encounter participant 228 (as audio acquisition devices 204, 206 are pointed to (i.e., directed toward) encounter participant 228). Additionally, modular ACD system 54 and/or audio recording system 104 may be configured to utilize audio acquisition devices 212, 214 to form audio recording beam 224, thus enabling the capturing of audio (e.g., speech) produced by encounter participant 230 (as audio acquisition devices 212, 214 are pointed to (i.e., directed toward) encounter participant 230). Further, modular ACD system 54 and/or audio recording system 104 may be configured to utilize null-steering precoding to cancel interference between speakers and/or noise.

As is known in the art, null-steering precoding is a method of spatial signal processing by which a multiple antenna transmitter may null multiuser interference signals in wireless communications, wherein null-steering precoding may mitigate the impact off background noise and unknown user interference.

In particular, null-steering precoding may be a method of beamforming for narrowband signals that may compensate for delays of receiving signals from a specific source at different elements of an antenna array. In general and to improve performance of the antenna array, in incoming signals may be summed and averaged, wherein certain signals may be weighted and compensation may be made for signal delays.

Machine vision system 100 and audio recording system 104 may be stand-alone devices (as shown in FIG. 2). Additionally/alternatively, machine vision system 100 and audio recording system 104 may be combined into one package to form mixed-media ACD device 232. For example, mixed-media ACD device 232 may be configured to be mounted to a structure (e.g., a wall, a ceiling, a beam, a column) within the above-described clinical environments (e.g., a doctor's office, a medical facility, a medical practice, a medical lab, an urgent care facility, a medical clinic, an emergency room, an operating room, a hospital, a long term care facility, a rehabilitation facility, a nursing home, and a hospice facility), thus allowing for easy installation of the same. Further, modular ACD system 54 may be configured to include a plurality of mixed-media ACD devices (e.g., mixed-media ACD device 232) when the above-described clinical environment is larger or a higher level of resolution is desired.

Additionally, modular ACD system 54 may be further configured to steer the one or more audio recording beams (e.g., audio recording beams 220, 222, 224) toward one or more encounter participants (e.g., encounter participants 226, 228, 230) of the patient encounter based, at least in part, upon machine vision encounter information 102. As discussed above, mixed-media ACD device 232 (and machine vision system 100/audio recording system 104 included therein) may be configured to monitor one or more encounter participants (e.g., encounter participants 226, 228, 230) of a patient encounter.

Specifically and as will be discussed below in greater detail, machine vision system 100 (either as a stand-alone system or as a component of mixed-media ACD device 232) may be configured to detect humanoid shapes within the above-described clinical environments (e.g., a doctor's office, a medical facility, a medical practice, a medical lab, an urgent care facility, a medical clinic, an emergency room, an operating room, a hospital, a long term care facility, a rehabilitation facility, a nursing home, and a hospice facility). And when these humanoid shapes are detected by machine vision system 100, modular ACD system 54 and/or audio recording system 104 may be configured to utilize one or more of the discrete audio acquisition devices (e.g., audio acquisition devices 202, 204, 206, 208, 210, 212, 214, 216, 218) to form an audio recording beam (e.g., audio recording beams 220, 222, 224) that is directed toward each of the detected humanoid shapes (e.g., encounter participants 226, 228, 230).

As discussed above, ACD compute system 12 may be configured to receive machine vision encounter information 102 and audio encounter information 106 from machine vision system 100 and audio recording system 104 (respectively); and may be configured to provide visual information 110 and audio information 114 to display rendering system 108 and audio rendering system 112 (respectively). Depending upon the manner in which modular ACD system 54 (and/or mixed-media ACD device 232) is configured, ACD compute system 12 may be included within mixed-media ACD device 232 or external to mixed-media ACD device 232.

### The Telehealth Assistance Process:

As mentioned above, when to comes to a telehealth appointment, a healthcare professional may be jeopardized by discoordination, technical difficulties, and/or challenges in communication and appointment administration—all of which may occur before and/or during a telehealth appointment—between a patient and a medical professional. When a patient visits a physical waiting room of a medical professional, information from the patient may be captured by an administrative assistant, nurse, or by the patient themselves (e.g., the patient may be asked to complete a paper form) all before the patient's session with a medical professional begins. While the patient may also provide the information via an automated kiosk and/or a questionnaire before the appointment, it may be beneficial to utilize time before a patient's session on a telehealth platform (e.g., telehealth platform 500) begins, at least because the above information from the patient may exclude important information that may be leveraged during the appointment with the medical professional.

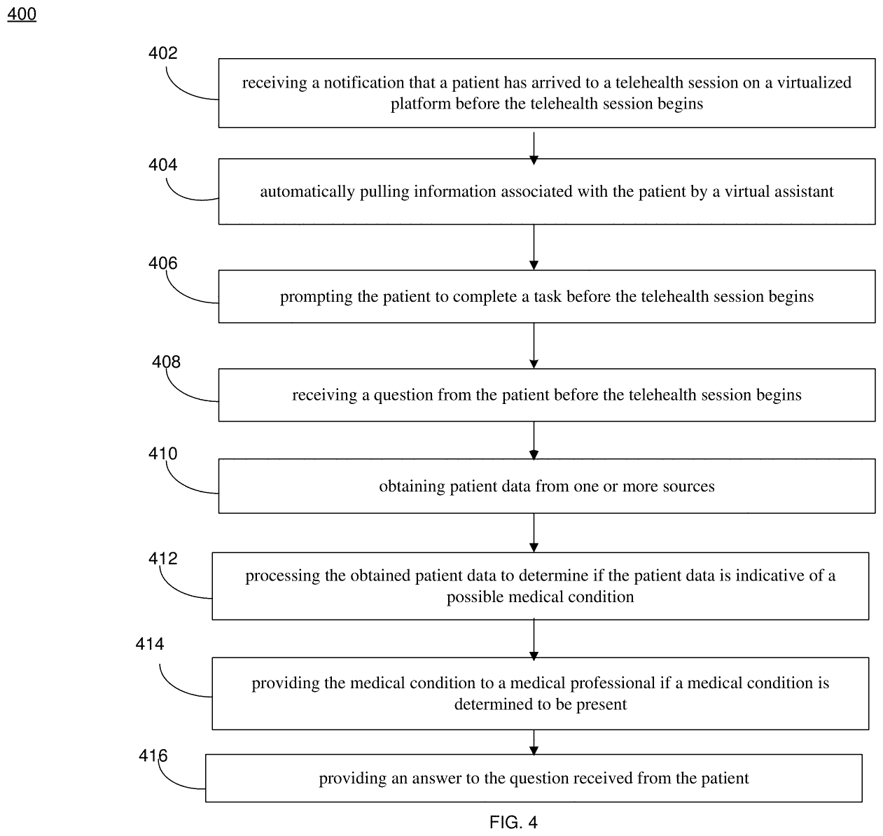

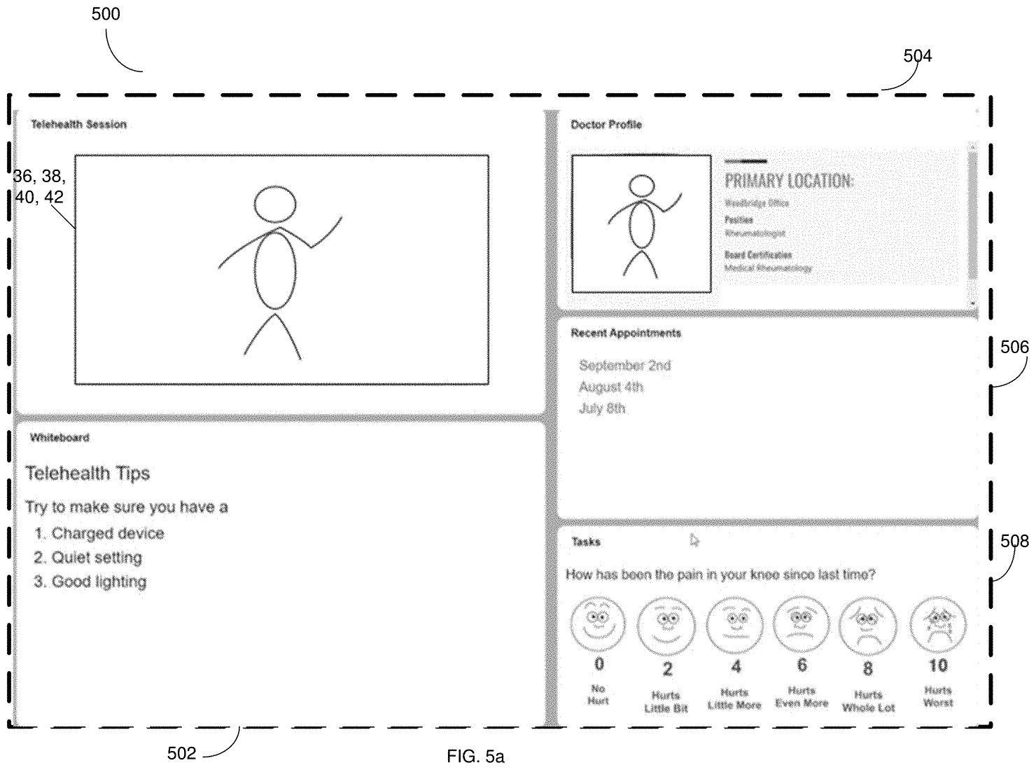

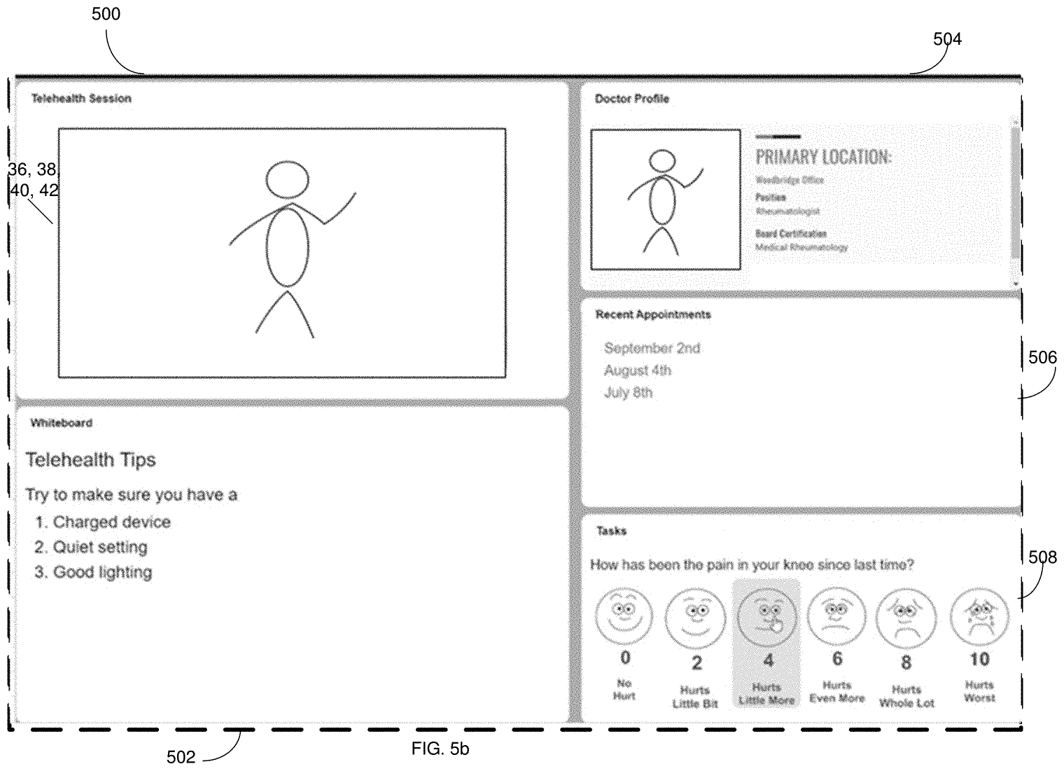

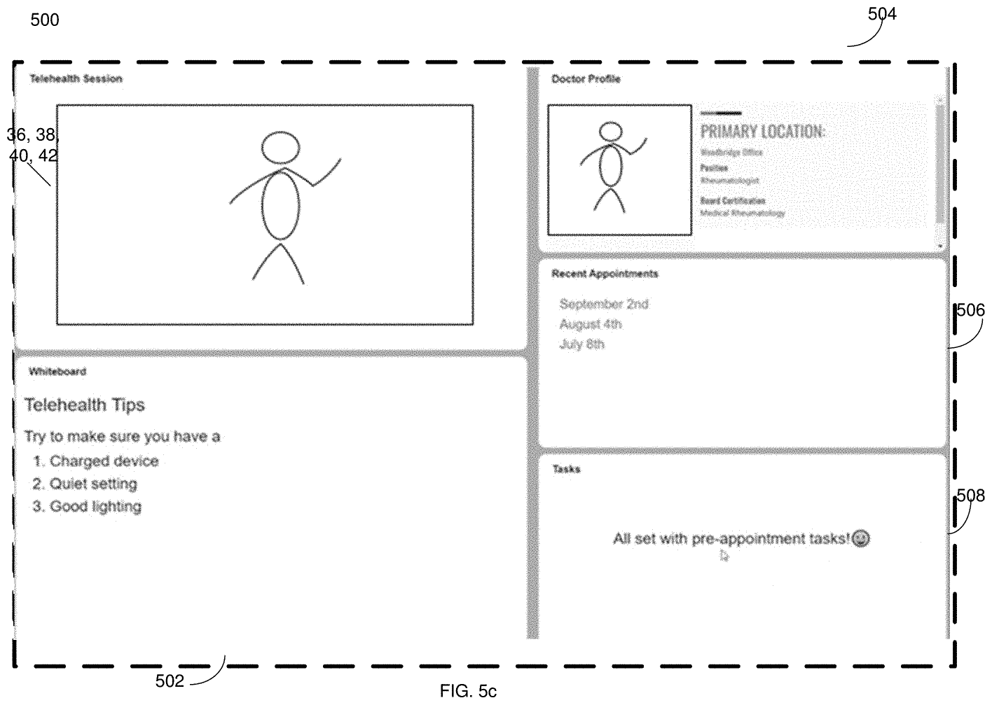

As discussed above and referring also at least to the example implementations of FIGS. 4-7, telehealth assistance process 10 may receive 402 a notification that a patient has arrived to a telehealth session on a virtualized platform (e.g., telehealth platform 500) before a telehealth session begins. For example, the patient may be a user (e.g., user 36, 38, 40, 42) who accesses the telehealth platform (e.g., telehealth platform 500) before a telehealth session is scheduled to begin, at a specific time the telehealth session is scheduled to begin, or after the time the telehealth session is scheduled to begin. Once the patient accesses the telehealth platform (e.g., telehealth platform 500), the patient may be placed in a virtual waiting room where the patient may interact with a virtual assistant (e.g., virtual assistant 238). The virtual assistant (e.g., virtual assistant 238) may interact with the patient via various interactive displays presented to the patient (e.g., display 502, 504, 506, 508). Further, use of the virtual assistant (e.g., virtual assistant 238) may include example and non-limiting benefits such as providing the medical professional with information, as described in more detail below, regarding the patient before the patient's telehealth session begins in order to allow for the medical professional to spend more time focusing on the patient during the telehealth session.

Additionally, once the notification that the patient has arrived to the telehealth session has been received 402, information associated with the patient may be automatically pulled 404 by the virtual assistant (e.g., via telehealth assistance process 10). The patient may then be prompted 406 by the virtual assistant (e.g., virtual assistant 238 via telehealth assistance process 10) to complete a task before the telehealth session begins. For example, the virtual assistant (e.g., virtual assistant 238) may prompt the patient for certain information such as an update to the patient's insurance information, a follow-up question from a previous appointment, collect a new concern from the patient, collect a change from the last appointment, and/or collect a current medial state of the patient utilizing, for example and not to be construed as a limitation, an interactive symptom checker such as an interactive scale (e.g., an interactive pain scale, as illustrated in display section 508). Further, the prompt from the virtual assistant (e.g., virtual assistant 238) may be presented/displayed to the patient in an interactive form such as a fillable form. Once the patient responds to the prompt from the virtual assistant (e.g., virtual assistant 238), the virtual assistant (e.g., virtual assistant 238) may notify the user that the patient has completed the task. For example, in response to the patient selecting a current pain level on an interactive pain scale on display 508, virtual assistant (e.g., virtual assistant 238) may display, e.g., “All set with pre-appointment tasks” on display 508. Additionally, a question may be received 408 (e.g., via telehealth assistance process 10) from the patient before the telehealth session beings. For example, the patient may be able to ask the virtual assistant (e.g., virtual assistant 238) via a text chat box in a display (e.g., display 502, 504, 506, 508) and/or speech recognition system associated with machine vision system 100 and/or audio recording system 104, a question about a previous visit with the medical professional, ask to review information about the medical professional their appointment is with, ask for a tip on how to best utilize the telehealth session, ask whether the medical professional the patient will see can prescribe a prescription, ask to see lab results, and ask if a lab appointment can be scheduled during the telehealth session with the medical professional and, if not, who to contact for a lab appointment. In order to answer the patient's question, the virtual assistant (e.g., virtual assistant 238) may access one or more data sources, as described below in more detail. For example, virtual assistant (e.g., virtual assistant 238) may display the answer to the patient's question in a display (e.g., display 502, 504, 506, 508). The virtual assistant (e.g., virtual assistant 238) may also help answer a frequently asked question (FAQ), but personalize the response for the patient. For example, the patient may ask the virtual assistant (e.g., virtual assistant 238), via a text chat box, about the cost of the visit with the medical professional. The virtual assistant (e.g., virtual assistant 238) may respond to the patient with a cost of the visit, which combines the type of visit with insurance information of the patient in order to provide a correct cost of the visit for the patient. In this example, the virtual assistant (e.g., virtual assistant 238 via telehealth assistance process 10) may automatically pull 404, for example and not to be construed as a limitation, the insurance information of the patient at the time the patient asks the question or the virtual assistant may have proactively pulled the insurance information associated with the patient with or without other information associated with the patient when the patient entered the telehealth session.

Further, patient data may be obtained 410 (e.g., via telehealth assistance process 10) from one or more sources. The one or more sources may include a patient database, machine vision system 100, and/or audio recording system 104. As explained in more detail above, machine vision system 100 may be configured to provide an analysis of a physical attribute of the patient and audio recording system 104 may provide an analysis of an auditory attribute of the patient. The physical attribute and/or auditory attribute may be obtained via a camera and/or microphone associated with the device the patient is using (e.g., ACD client electronic devices 28, 30, 32, 34). The obtained physical attribute and/or auditory attribute may be provided to the virtual assistant (e.g., virtual assistant 238).

Additionally, a complete recording of the telehealth session (e.g., audio/video from the user's computing device and/or the medical professional's computing device), including any time spent on the telehealth platform by the user before the telehealth session begins and/or after the telehealth session begins, may be generated in the form of, for example but not limited to, an encounter transcript (e.g., encounter transcript 234) from utilizing machine vision system 100 and/or audio recording system 104. At least a portion of this encounter transcript (e.g., encounter transcript 234) may be processed to populate at least a portion of a medical record (e.g., medical record 236) associated with the telehealth session.

As discussed above, ACD compute system 12 may be configured to access one or more datasources (e.g., datasources 118), wherein examples of datasources 118 may include a medical conditions symptoms datasource (e.g., that defines the symptoms for various diseases and medical conditions, including but not limited to skin color as indications of potential medical conditions), a prescriptions compatibility datasource (e.g., that defines groups of prescriptions that are substitutable for (or compatible with) each other), a medical insurance coverage datasource (e.g., that defines what prescriptions are covered by various medical insurance providers), and a home healthcare datasource (e.g., that defines best practices concerning when home healthcare is advisable). Accordingly, telehealth assistance process 10 may process the data included within the encounter information (e.g., machine vision encounter information 102 and/or audio encounter information 106) to compare this data to data defined within the datasources (e.g., datasources 118) to determine if the encounter information (e.g., machine vision encounter information 102 and/or audio encounter information 106) is indicative of a potential medical situation.

The obtained data may be processed 412 (e.g., via telehealth assistance process 10) to determine if the patient data is indicative of a possible medical condition. For example, the obtained data may include, as described below, an audio recording of the patient coughing (whether intentionally asked for by the medical professional or by necessity of the user actually needing to cough). The cough may be recorded after the patient accesses the telehealth platform (e.g., telehealth platform 500) before the telehealth session begins or during the telehealth session. The cough may be processed and analyzed against the one or more data sources described below. For example, the cough may be analyzed and compared to a cough in a data source as described below associated with a known condition such as, for example, pneumonia. Alternatively, the obtained data may include an analysis of the patient's voice recorded after the patient accesses the telehealth platform (e.g., telehealth platform 500) before the telehealth session begins or during the telehealth session. The patient's voice recording may be compared to a voice recording in the one or more data sources described below. For example, the patient's voice recording may be compared to a voice recording associated with a common COVID-19. Further, the obtained data may include a color of the patient's skin, as described below. The patient's skin color may be analyzed after the patient accesses the telehealth platform (e.g., telehealth platform 500) before the telehealth session begins or during the telehealth session. For example, the patient's skin color may have a yellowish/jaundiced complexion and that skin color may be compared to any of the above-noted datasources. If a medical condition is determined to be present, the medical condition may be provided 414 (e.g., via telehealth assistance process 10) to the medical professional. Referring to the above example, if the patient's cough matches a cough associated with pneumonia, pneumonia may be identified as a possible medical condition and provided to the medical professional. Furtherer, if the patient's voice recording is compared to a voice recording associated with COVID-19 and COVID-19 is identified as a possible medical condition, the medical condition may be provided to the medical professional. The medical condition may include, but is not limited to, one or more of: a potential medical condition; a potential medication issue; a potential home healthcare issue; and a potential follow-up issue.

Additionally, the virtual assistant (e.g., virtual assistant 238) may provide 416 (e.g., telehealth assistance process 10) an answer to the question received from the patient. The answer may be personalized to the patient. For example, if the patient asked about an estimated cost of the telehealth session with the medical professional, the virtual assistant (e.g., virtual assistant 238) may combine the patient's obtained insurance information with details of the telehealth session to provide the patient with an estimated cost of the telehealth session with the medical professional.

In some embodiments, the obtained patient data may be compared to past patient data recorded or otherwise obtained from one or more previous telehealth visits or otherwise previously/currently recorded or detailed in the medical records of the patient. It may then be determined if a medical condition is present and if a previously identified medical condition is present based at least in part on the obtained patient data and the past patient data.

In some embodiments, the physical attribute of the patient may include an analysis by the virtual assistant (e.g., virtual assistant 238 via telehealth assistance process 10) of the patient's skin color by the virtual assistant to determine the patient's blood pressure or other potential ailments that may be determined by the patient's skin. For instance, skin color may be used as an additional indicator of overall health. For example, a patient's skin color may appear dark in color, which may be indicative of high blood pressure. As another example, pallor (pale/deficiency of color) may indicate anemia, cyanosis (bluish discoloration) may signal hypoxemia, etc. The virtual assistant (e.g., virtual assistant 238 via telehealth assistance process 10) may compare the patient's dark skin color to a skin color captured from the patient during a previous visit (e.g., to see if there is a change or if this is simply the patient's normal skin color) and/or to a data source as described above to indicate a potential medical condition.

In some embodiments, the auditory attribute of the patient may include a cough recorded by audio recording system after the patient has arrived to the telehealth session and before the telehealth session begins. For example, and not to be construed as a limitation, the virtual assistant may prompt the patient to cough (or the patient may naturally cough on their own without a prompt) and the audio of the cough may be recorded and analyzed to determine if a possible medical condition is present, or an alert may be provided to the medical professional indicating that the patient coughed (e.g., with a recording of the cough played back to the medical professional).

In some embodiments, if the virtual assistant determines it cannot answer a question from the patient (e.g., because there is insufficient information to answer the question or the question is not understandable to the virtual assistant), the virtual assistant may provide the question to the medical professional at the start of the telehealth session. The question may be provided, for example and not to be construed as a limitation, in the form of a list. The question may be provided via, e.g., email, text, or on the display used by the medical professional.

In some implementations, the virtual assistant (e.g., virtual assistant 238) may be integrated with a platform such as the Dragon® Ambient eXperience™ (DAX) platform and/or the Electronic Health Record (EHR) platform provided by Nuance Communications, Inc. Further, the virtual assistant (e.g., virtual assistant 238) may be paired with a real-time speech to text transcription system such as Krypton, provided by Nuance Communications, Inc., via a patient interface/portal.

### General:

As will be appreciated by one skilled in the art, the present disclosure may be embodied as a method, a system, or a computer program product. Accordingly, the present disclosure may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, the present disclosure may take the form of a computer program product on a computer-usable storage medium having computer-usable program code embodied in the medium.

Any suitable computer usable or computer readable medium may be utilized. The computer-usable or computer-readable medium may be, for example but not limited to, an electronic, magnetic, optical, electromagnetic, infrared, or semiconductor system, apparatus, device, or propagation medium. More specific examples (a non-exhaustive list) of the computer-readable medium may include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a transmission media such as those supporting the Internet or an intranet, or a magnetic storage device. The computer-usable or computer-readable medium may also be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory. In the context of this document, a computer-usable or computer-readable medium may be any medium that can contain, store, communicate, propagate, or transport the program for use by or in connection with the instruction execution system, apparatus, or device. The computer-usable medium may include a propagated data signal with the computer-usable program code embodied therewith, either in baseband or as part of a carrier wave. The computer usable program code may be transmitted using any appropriate medium, including but not limited to the Internet, wireline, optical fiber cable, RF, etc.

Computer program code for carrying out operations of the present disclosure may be written in an object-oriented programming language such as Java, Smalltalk, C++ or the like. However, the computer program code for carrying out operations of the present disclosure may also be written in conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through a local area network/a wide area network/the Internet (e.g., network 14).

The present disclosure is described with reference to flowchart illustrations and/or block diagrams of methods, apparatus (systems) and computer program products according to embodiments of the disclosure. It will be understood that each block of the flowchart illustrations and/or block diagrams, and combinations of blocks in the flowchart illustrations and/or block diagrams, may be implemented by computer program instructions. These computer program instructions may be provided to a processor of a general-purpose computer/special purpose computer/other programmable data processing apparatus, such that the instructions, which execute via the processor of the computer or other programmable data processing apparatus, create means for implementing the functions/acts specified in the flowchart and/or block diagram block or blocks.

These computer program instructions may also be stored in a computer-readable memory that may direct a computer or other programmable data processing apparatus to function in a particular manner, such that the instructions stored in the computer-readable memory produce an article of manufacture including instruction means which implement the function/act specified in the flowchart and/or block diagram block or blocks.

The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions/acts specified in the flowchart and/or block diagram block or blocks.

The flowcharts and block diagrams in the figures may illustrate the architecture, functionality, and operation of possible implementations of systems, methods and computer program products according to various embodiments of the present disclosure. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustrations, and combinations of blocks in the block diagrams and/or flowchart illustrations, may be implemented by special purpose hardware-based systems that perform the specified functions or acts, or combinations of special purpose hardware and computer instructions.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present disclosure has been presented for purposes of illustration and description, but is not intended to be exhaustive or limited to the disclosure in the form disclosed. Many modifications and variations will be apparent to those of ordinary skill in the art without departing from the scope and spirit of the disclosure. The embodiment was chosen and described in order to best explain the principles of the disclosure and the practical application, and to enable others of ordinary skill in the art to understand the disclosure for various embodiments with various modifications as are suited to the particular use contemplated.

A number of implementations have been described. Having thus described the disclosure of the present application in detail and by reference to embodiments thereof, it will be apparent that modifications and variations are possible without departing from the scope of the disclosure defined in the appended claims.

What is claimed is:

1. A computer-implemented method comprising:
receiving, via a computing device, a notification that a patient has arrived to a telehealth session on a virtualized platform before the telehealth session begins;
automatically, in response to receiving the notification that the patient has arrived to the telehealth session before the telehealth session begins, pulling information associated with the patient by a virtual assistant;
prompting, via the virtual assistant, the patient to complete a task before the telehealth session begins;
receiving a question from the patient before the telehealth session begins;
obtaining patient data from one or more sources, the one or more sources including at least one of a patient database, a machine vision system that is configured to provide an analysis of a physical attribute of the patient, and an audio recording system that is configured to provide an analysis of an auditory attribute of the patient;
processing the obtained patient data to determine if the patient data is indicative of a possible medical condition;
if a medical condition is determined to be present, providing the medical condition to a medical professional; and
providing an answer to the question received from the patient, wherein the answer is personalized to the patient.

2. The computer-implemented method of claim 1, wherein the task to be completed by the patient before the telehealth session begins includes one or more of a fillable form and an interactive symptom checker.

3. The computer-implemented method of claim 2, wherein the interactive symptom checker includes an interactive scale where the patient indicates a current physical pain level.

4. The computer-implemented method of claim 1, wherein the question the patient asks the virtual assistant includes one or more of a question regarding which medical provider the telehealth appointment will be with, a question regarding the patient's insurance, and a question regarding past patient data recorded during a previous telehealth visit.

5. The computer-implemented method of claim 4, further comprising:
comparing the obtained patient data to the past patient data recorded in the previous telehealth visit;
determining, based at least in part on the obtained patient data and the past patient data, one or more of if a medical condition is present and if a previously identified medical condition is present.

6. The computer-implemented method of claim 1, wherein the auditory attribute of the patient includes a cough recorded by the audio recording system after the patient has arrived to the telehealth session and before the telehealth session begins.

7. The computer-implemented method of claim 1, further comprising:
determining that the virtual assistant cannot answer the question from the patient; and
in response to determining that the virtual assistant cannot answer the question from the patient, providing the question to the medical professional when the telehealth session begins.

8. A computer program product residing on a computer readable medium having a plurality of instructions stored thereon which, when executed by a processor, cause the processor to perform operations comprising:
receiving, via a computing device, a notification that a patient has arrived to a telehealth session on a virtualized platform before the telehealth session begins;
automatically, in response to receiving the notification that the patient has arrived to the telehealth session before the telehealth session begins, pulling information associated with the patient by a virtual assistant;
prompting, via the virtual assistant, the patient to complete a task before the telehealth session begins;
receiving a question from the patient before the telehealth session begins;
obtaining patient data from one or more sources, the one or more sources including at least one of a patient database, a machine vision system that is configured to provide an analysis of a physical attribute of the patient, and an audio recording system that is configured to provide an analysis of an auditory attribute of the patient;
processing the obtained patient data to determine if the patient data is indicative of a possible medical condition;
if a medical condition is determined to be present, providing the medical condition to a medical professional; and
providing an answer to the question received from the patient, wherein the answer is personalized to the patient.

9. The computer program product of claim 8, wherein the task to be completed by the patient before the telehealth session begins includes one or more of a fillable form and an interactive symptom checker.

10. The computer program product of claim 8, wherein the interactive symptom checker includes an interactive scale where the patient indicates a current physical pain level.

11. The computer program product of claim 8, wherein the question the patient asks the virtual assistant includes one or more of a question regarding which medical provider the telehealth appointment will be with, a question regarding the patient's insurance, and a question regarding past patient data recorded during a previous telehealth visit.

12. The computer program product of claim 11, further comprising:
comparing the obtained patient data to the past patient data recorded in the previous telehealth visit;
determining, based at least in part on the obtained patient data and the past patient data, one or more of if a medical condition is present and if a previously identified medical condition is present.

13. The computer program product of claim 8, wherein the auditory attribute of the patient includes a cough recorded by the audio recording system after the patient has arrived to the telehealth session and before the telehealth session begins.

14. The computer program product of claim 8, further comprising:
determining that the virtual assistant cannot answer the question from the patient; and
in response to determining that the virtual assistant cannot answer the question from the patient, providing the question to the medical professional when the telehealth session begins.

15. A computing system including a processor and memory configured to perform operations comprising:
receiving, via a computing device, a notification that a patient has arrived to a telehealth session on a virtualized platform before the telehealth session begins;
automatically, in response to receiving the notification that the patient has arrived to the telehealth session before the telehealth session begins, pulling information associated with the patient by a virtual assistant;
prompting, via the virtual assistant, the patient to complete a task before the telehealth session begins;
receiving a question from the patient before the telehealth session begins;
obtaining patient data from one or more sources, the one or more sources including at least one of a patient database, a machine vision system that is configured to provide an analysis of a physical attribute of the patient, and an audio recording system that is configured to provide an analysis of an auditory attribute of the patient;
processing the obtained patient data to determine if the patient data is indicative of a possible medical condition;
if a medical condition is determined to be present, providing the medical condition to a medical professional; and
providing an answer to the question received from the patient, wherein the answer is personalized to the patient.

16. The computing system of claim 15, wherein the task to be completed by the patient before the telehealth session begins includes one or more of a fillable form and an interactive symptom checker.

17. The computing system of claim 15, wherein the interactive symptom checker includes an interactive scale where the patient indicates a current physical pain level.

18. The computing system of claim 15, wherein the question the patient asks the virtual assistant includes one or more of a question regarding which medical provider the telehealth appointment will be with, a question regarding the patient's insurance, and a question regarding past patient data recorded during a previous telehealth visit.

19. The computing system of claim 18, further comprising:
comparing the obtained patient data to the past patient data recorded in the previous telehealth visit;
determining, based at least in part on the obtained patient data and the past patient data, one or more of if a medical condition is present and if a previously identified medical condition is present.

20. The computing system of claim 15, wherein the auditory attribute of the patient includes a cough recorded by the audio recording system after the patient has arrived to the telehealth session and before the telehealth session begins.

21. The computing system of claim 15, further comprising:
determining that the virtual assistant cannot answer the question from the patient; and
in response to determining that the virtual assistant cannot answer the question from the patient, providing the question to the medical professional when the telehealth session begins.
